(19) United States
(12) Patent Application Publication
(10) Pub. No.: US 2015/0073388 A1
(43) Pub. Date: Mar. 12, 2015

(54) DRUG DELIVERY SYSTEMS AND RELATED METHODS

(71) Applicants: Maria PALASIS, Wellesley, MA (US); Danny CONCAGH, Medfield, MA (US); Lee CORE, Needham, MA (US); Stephanie WEBBER, Brookline, MA (US); Yina KUANG, Newton, MA (US); Wendy NAIMARK, Boston, MA (US); Raymond Knox, Worcester, MA (US)

(72) Inventors: Maria PALASIS, Wellesley, MA (US); Danny CONCAGH, Medfield, MA (US); Lee CORE, Needham, MA (US); Stephanie WEBBER, Brookline, MA (US); Yina KUANG, Newton, MA (US); Wendy NAIMARK, Boston, MA (US); Raymond Knox, Worcester, MA (US)

(21) Appl. No.: 14/481,583

(22) Filed: Sep. 9, 2014

Related U.S. Application Data
(60) Provisional application No. 61/875,357, filed on Sep. 9, 2013.

(51) Int. Cl. A61M 25/10; A61M 25/00

(52) U.S. Cl. CPC A61M 25/1002; A61M 25/002; A61M 2025/1052; A61M 2025/1054; A61M 2025/1075; A61M 2025/105
USPC 604/509; 604/103.02

(57) ABSTRACT

The present invention provides systems and methods for the targeted delivery of a therapeutic agent using expandable devices that are temporarily or permanently placed within the body. Such therapeutic agents may be delivered as a coating on the outer surface of the expandable device. Once positioned at a desired location within the body, the device is expanded such that its drug-coated outer surface establishes direct contact with a target tissue. The expanded device remains in contact with the target tissue for a pre-determined amount of time prior to being withdrawn from the body. Delivery of the therapeutic agent using the expandable device may be followed, or preceded, by delivery of a second expandable device to the same location within the body.

### CROSS REFERENCE TO RELATED APPLICATIONS

This application claims priority to and the benefit of U.S. Provisional Application Ser. No. 61/875,357, filed on Sep. 9, 2013, herein incorporated by reference in its entirety.

### FIELD OF THE INVENTION

The present invention relates generally to systems and methods for delivering therapeutic agents to specific locations within the body. In particular, the present invention relates to the targeted delivery of therapeutic agents loaded onto expandable devices that are temporarily or permanently placed within the body.

### BACKGROUND OF THE INVENTION

Therapeutic agents are locally delivered to various locations in the human body for a variety of clinical applications. For example, therapeutic agents may be delivered to locations within the cardiovascular, gastrointestinal, urinary and other systems to treat diseased tissue or other conditions. Such therapeutic agents may be delivered by delivery systems that include, for example, platforms that are temporarily placed or permanently implanted into the body.

Drug delivery systems that utilize expandable balloons are known in the art. By way of non-limiting example, drug eluting balloons (“DEBs”) such as the IN.PACT Drug Eluting Balloon (Medtronic, Minneapolis, Minn.) and the Lutonix drug-coated balloon (“DCB”) (Lutonix, Inc., New Hope, Minn.) are available for the delivery of paclitaxel into diseased blood vessels for the prevention of restenosis. Such systems are based upon percutaneous transluminal angioplasty (“PTA”) balloons coated with a matrix that includes paclitaxel. These balloons are available in 2.5-4.0 mm diameter (for coronary indications) and 6-7 mm diameter (for peripheral indications) sizes, with lengths that range up to 120 mm.

In use, a DEB is delivered to a target location within a blood vessel via a delivery catheter, whereupon the DEB is extruded from the delivery catheter and inflated such that its outer surface comes into contact with the blood vessel wall. The drug is delivered from the coating matrix while in contact with the blood vessel wall. After a short therapy time (i.e., on the order of seconds or minutes), the balloon is deflated, pulled back into the delivery catheter, and removed from the body.

Although clinical trials using DEBs have shown promising results for the reduction of target lesion revascularization and restenosis when compared with the use of uncoated PTA balloons, the use and effectiveness of DEBs may be limited by the acute nature of the therapy. That is, since DEBs only deliver drug at the time of use they are incapable of administering drugs for an extended time period. After the DEB is deflated and removed from the body, the only drug remaining at the target location is the drug that had been absorbed by the surrounding tissue during the short time that the inflated balloon was in contact with the blood vessel wall. The utility of DEBs may therefore be limited to procedures that do not require long-term drug delivery. What is needed is a delivery system that allows for prolonged exposure of therapeutic agents to specific locations within the body.

### SUMMARY OF THE INVENTION

In one aspect, the present invention addresses the shortcomings of current DEB technologies and significantly increases drug retention within tissues following drug delivery via DEBs. Advantages of the present invention include the ability to significantly reduce the concentration of drug(s) loaded within DEBs, and the ability to use DEBs as part of a system for long-term local drug delivery to blood vessels and other bodily lumens.

In one aspect, the present invention comprises a system that includes a first expandable device and a second expandable device. The first expandable device, such as a DEB, has an outer surface that is at least partially coated with, or is otherwise configured for the delivery of, a therapeutic agent, such as paclitaxel. The second expandable device comprises a tubular structure comprising a braided structure of at least one strand. While the surface coverage may change based on the size of the vessel in which they are deployed—in one embodiment having a manufactured diameter of 7 mm—the braided structure is characterized by an as-manufactured surface area of at least 100 square millimeters per 10 millimeters of tubular structure length.

In another aspect, the present invention comprises a method of treating a patient. For example, in one embodiment the method comprises the steps of introducing a first expandable device into a blood vessel, contacting an internal surface of the blood vessel with at least a portion of an outer surface of the first expandable device, delivering a therapeutic agent to the internal surface of the blood vessel from the outer surface of the first expandable device, and withdrawing the first expandable device from the patient. The method also includes the steps of introducing a second expandable device into a blood vessel, expanding the second expandable device in the blood vessel, contacting the internal surface of the blood vessel with a surface of the second expandable device, and leaving the second expandable device permanently implanted in the blood vessel. In certain embodiments, the first expandable device is introduced into and withdrawn from the blood vessel before the second expandable device is introduced into the blood vessel. In other embodiments, the first expandable device is introduced into and withdrawn from the blood vessel after the second expandable device is introduced into and permanently implanted into the blood vessel.

In yet another aspect, the present invention includes a kit that comprises the components of the systems described herein.

In yet another aspect, the present invention comprises a set of instructions that educates or instructs a physician or other healthcare provider to use the systems and kits of the present invention in accordance with the methods described herein.

### BRIEF DESCRIPTION OF THE DRAWINGS

Non-limiting embodiments of the present invention will be described by way of example, with reference to the accompanying figures, which are schematic in nature and are not intended to be drawn to scale. In the figures, each identical or nearly identical component illustrated in typically represent by a single numeral. For purposes of clarity, not every component is labeled in every figure, nor is every component of each embodiment of the invention shown where illustration is not necessary to allow those of ordinary skill in the art to understand the invention. In the figures:

FIG. 1 depicts a DEB positioned within the lumen of a blood vessel, in accordance with an embodiment of the present invention.

FIG. 2 is a cross-sectional view of a DEB, in accordance with an embodiment of the present invention.

FIG. 3 depicts a second expandable device, in accordance with an embodiment of the present invention.

### DETAILED DESCRIPTION OF THE PREFERRED EMBODIMENTS

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

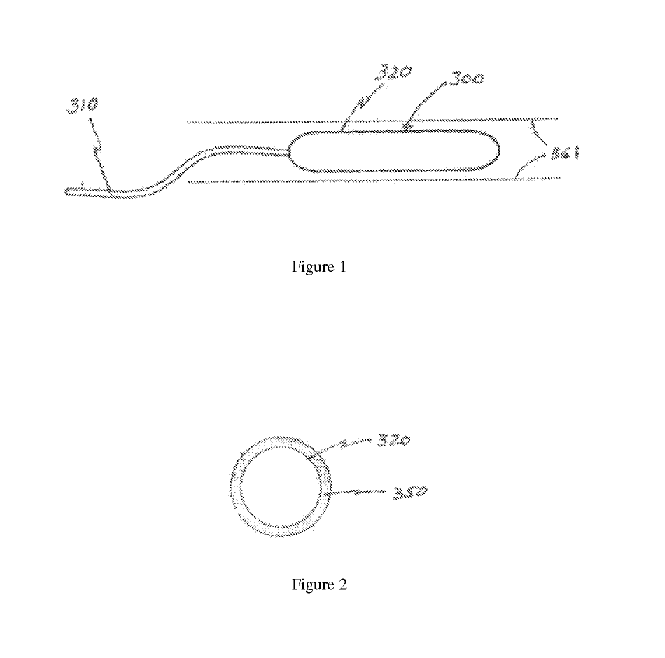

In a preferred embodiment, the first expandable device of the present invention is a drug coated balloon 300, as shown in FIG. 1. As used herein “drug coated balloon” is used synonymously with “drug eluting balloon” or “DEB” to refer to an expandable bladder at the distal end of a catheter 310 or similar device, which is either coated with or otherwise elutes or delivers a therapeutic agent from or through its outer surface 320.

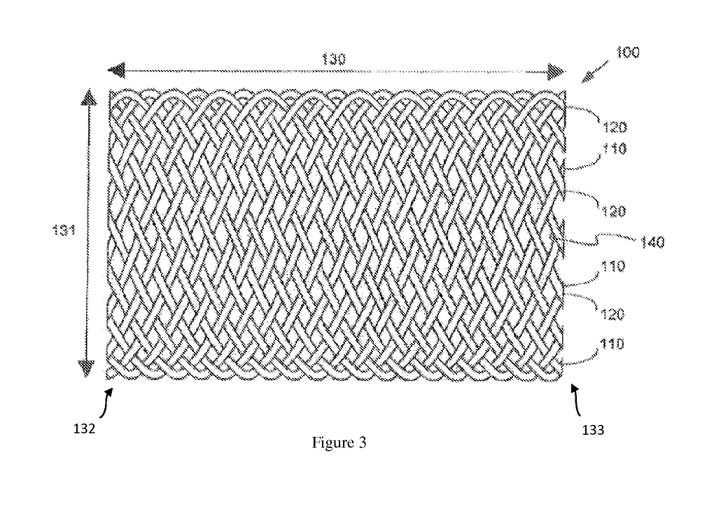

As shown in cross-section in FIG. 2, DEBs include, in a preferred embodiment, a coating 350 that incorporates a therapeutic agent to be delivered by the DEB to surrounding tissue(s). The coating 350 comprises any suitable polymer, gel, foam or other matrix material that can retain the therapeutic agent until the DEB is delivered to a target site within a blood vessel or other bodily lumen 360, and then elute the agent from the coating 350 when the balloon comes into contact with the wall 361 of the blood vessel or other bodily lumen. As used herein “elute” is used as a general term to refer to any mechanism by which a therapeutic agent is transferred from the DEB into the area(s) around the DEB, including physical transfer not requiring dissolution of the drug. In other embodiments, the DEB does not include a coating but instead infuses or otherwise transmits a drug solution through the outer surface 320 of DEB 300 via pores or holes (arranged randomly or in a pattern) or otherwise. In yet other embodiments, the therapeutic agent is in particulate or other solid form and is applied directly onto the outer surface 320 of DEB 300 in the absence of a carrier matrix.

In one aspect, the coating 350 optionally comprises excipients and/or other additives to give the coating 350 hydrophilic or other desired properties, and/or to enhance the elution of drug from the coating 350 and/or the absorption of the drug into surrounding tissues. In one embodiment, the coating 350 comprises urea.

In one aspect, the DEB 300 is preferably of any suitable dimension(s) for treatment of a diseased body lumen or cavity. In a preferred embodiment, DEB 300 has a cross-sectional diameter of 6-7 mm and a length of 40, 60, 80, 100 or 120 mm. Such dimensions are particularly suitable for the treatment of the superficial femoral artery (“SFA”), arteries below-the-knee, and other peripheral arteries. In other embodiments, the DEB 300 is characterized by smaller sizes suitable for use within the coronary arteries, as is known in the art for PTA balloons.

In one aspect, the therapeutic agent eluted by the DEB 300 is determined by the medical condition to be treated. By way of non-limiting example, to treat or prevent restenosis the therapeutic agent is an anti-proliferative or other suitable agent as known in the art. In a preferred embodiment, the therapeutic agent is paclitaxel, its analogues, and/or its derivatives. In other embodiments, the therapeutic agent is everolimus, sirolimus, zotarolimus, biolimus or combinations thereof.

In one aspect, the second expandable device of the present invention is a polymeric implant as described in co-owned U.S. Pat. No. 8,137,396, which is herein incorporated in its entirety for all purposes. With reference to the second expandable device, as used herein the terms “device,” “implant,” and “stent” are used synonymously. Similarly, as used herein, the term “self-expanding” includes devices that are crimped to a reduced configuration for delivery into a bodily lumen or cavity, and thereafter expand to a larger suitable configuration once released from the delivery configuration. Such expansion may occur without the aid of additional expansion devices or with the partial aid of balloon-assisted or similarly-assisted expansion.

As depicted in FIG. 3, in one embodiment the second expandable device 100 preferably comprises at least one strand woven together to form a substantially tubular configuration having a longitudinal dimension 130, a radial dimension 131, and first and second ends 132, 133 along the longitudinal dimension. As used herein, “woven” is used synonymously with “braided.” For example, the tubular configuration may be woven to form a tubular structure comprising two sets of strands 110 and 120, with each set extending in an opposed helix configuration along the longitudinal dimension of the implant. The sets of strands 110 and 120 cross each other at a braid angle 140, which may be constant or may change along the longitudinal dimension of the implant. Preferably, between about 16 and about 96 strands are used in the implants of the present invention, and more preferably 16 to 32 strands; and the braid angle 140 is within the range of about 90 degrees to about 135 degrees throughout the implant, more preferably within a range of about 110 degrees to 135 degrees, and even more preferably within a range of about 115 degrees to 130 degrees. The strands are woven together using methods known in the art, including for example, weave patterns such as Regular pattern “1 wire, 2-over/2-under,” Diamond half load pattern “1 wire, 1-over/1-under,” or Diamond pattern “2 wire, 1-over/1-under.”

Although the strands may be made from biostable polymeric or metallic materials, they are preferably made from at least one biodegradable material that is preferably fully absorbed within about two years of placement within a patient, and more preferably within about one year of placement within a patient. In some embodiments, the strands are fully absorbed within about six or fewer months of placement within a patient. The first and second strand sets 110, 120 may be made from the same or different biodegradable polymer. Non-limiting examples of biodegradable polymers that are useful in the at least one strand of the present invention include poly lactic acid (PLA), poly glycolic acid (PGA), poly trimethylene carbonate (PTMC), poly caprolactone (PCL), poly dioxanone (PDO) and copolymers thereof. Preferred polymers are poly(lactic acid co-glycolic acid) (PLGA) having a weight percentage of up to about 20% lactic acid, or greater than about 75% lactic acid (preferably PLGA 85:15), with the former being stronger than the latter but degrading in the body faster. The composition of PLGA polymers within these ranges may be optimized to meet the mechanical property and degradation requirements of the specific application for which the implant is used. For desired expansion and mechanical property characteristics, the materials used for the strands preferably have an elastic modulus within the range of about 1 GPa to about 10 GPa, and more preferably within the range of about 6-10 GPa. In some embodiments, the strands used in the devices of the present invention are at least partially coated with a conformal coating of a suitable biocompatible material, preferably comprising a biodegradable elastomeric material such as poly(lactic acid-co-caprolactone).

To facilitate the low-profile aspects of the present invention (e.g., the delivery of the implants into small diameter bodily lumens or cavities), the strands used in the second expandable device 100 preferably have cross-sectional diameters in the range of from about 0.003 inches to about 0.007 inches. Where multiple strands are used they may be of substantially equal diameters within this range, or first strand set 110 may be of a different general diameter than second strand set 120. In either event, the diameters of strands are chosen so as to render the implant 100 preferably deliverable from a 10 French delivery catheter (i.e., 3.3 mm diameter) or smaller, and more preferably from a 7 French delivery catheter (i.e., 2.3 mm diameter) or smaller. The ability to place the devices of the present invention into small diameter delivery catheters allows for their implantation into small diameter bodily lumens and cavities, such as those found in the vascular, biliary, uro-genital, iliac, and tracheal-bronchial anatomy. Exemplary vascular applications include coronary as well as peripheral vascular placement, such as in the SFA. It should be appreciated, however, that the implants of the present invention are equally applicable to implantation into larger bodily lumens, such as those found in the gastrointestinal tract, and for applications such as esophageal scaffolds.

In a preferred embodiment, the second expandable device is comprised of 8 to 32 conformally coated strands having cross-sectional diameters in the range of 0.003-0.007 inches. More preferably, the second expandable device is configured with a pre-determined number of strands and cross-sectional diameter(s) to yield an implant with an overall surface area of at least about 100 square millimeters, more preferably at least about 200 square millimeters, more preferably at least about 250 square millimeters, more preferably at least about 300 square millimeters, and most preferably at least about 350 square millimeters; in each case measured per every 10 millimeters of the longitudinal length of the implant. In some embodiments, the surface area of the second expandable device is 100-350 square millimeters for every 10 millimeters of longitudinal length of the implant. In other embodiments, the surface area of the second expandable device is 200-500 square millimeters, and preferably 300-400 square millimeters for every 10 millimeters of the longitudinal length of the implant. It will be appreciated that reference to cross-sectional diameters is in no way limiting to strands (i.e., fibers) that are substantially round, but includes a variety of shapes, including for example, strands that are substantially flat. Table I sets forth the approximate surface area of embodiments of the second expandable device of the present invention. All surface area values are based on a uniform coating of the elastomer on the braided strand.

TABLE 1

| | Surface Area of Second Expandable | | | | |
|---|---|---|---|---|---|
| | Device (mm²/10 mm of device length) | | | | |
| | 0.003″ | 0.004″ | 0.005″ | 0.006″ | 0.007″ |
| # strands | diameter | diameter | diameter | diameter | diameter |
| 8 | 57 | 75.5 | 95 | 114 | 133 |
| 16 | 114 | 151 | 190 | 227 | 265 |
| 24 | 171 | 227 | 285 | 341 | 398 |
| 32 | 227 | 303 | 379 | 454 | 531 |

In some cases, the second expandable devices of the present invention utilize multiple strands of differing diameters. Preferred embodiments of the present invention make use of second expandable devices comprised of 16 strands at 0.006″ and 0.007″, 24 strands at 0.004″, 0.005″, 0.006″ and 0.007″, and 32 strands at 0.003″, 0.004″, 0.005″, 0.006″ and 0.007″; more preferred embodiments make use of second expandable devices comprised of 16 strands at 0.007″, 24 strands at 0.005″, 0.006″ and 0.007″, and 32 strands at 0.004″, 0.005″, 0.006″ and 0.007″; more preferred embodiments make use of second expandable devices comprised of 24 strands at 0.006″ and 0.007″, and 32 strands at 0.004″, 0.005″, 0.006″ and 0.007″; and most preferred embodiments make use of second expandable devices comprised of 24 strands at 0.007″, and 32 strands at 0.005″, 0.006″ and 0.007.″

As previously described, the use of DEBs for the treatment of diseased blood vessels is known in the art. Because the drug delivered by DEBs of the prior art are kept against a surrounding lumen wall only during the time in which the outer surface of the DEB is in contact with the lumen wall, conventional DEBs must be loaded with a relatively large quantity of drug in order to transfer as much drug as possible to tissue in as short a time period as possible. For example, conventional DEBs that are used to deliver paclitaxel to SFAs are loaded with 3 micrograms per square millimeter of balloon surface area, which translates to approximately 2,640 micrograms total for a 7 mm×40 mm balloon (which has a surface area of about 880 square millimeters) and approximately 2,260 micrograms for a 6 mm×40 mm balloon (which has a surface area of about 750 square millimeters).

In contrast to conventional DEBs, the first expandable device(s) of the present invention are able to be loaded with a significantly lower amount of therapeutic agent and yet, when used in conjunction with the second expandable devices of the present invention, result in long-term drug retention within affected tissues with commensurate long-term therapeutic effects. For example, in one embodiment of the present invention, the first expandable device is a DEB loaded with a therapeutic agent at a concentration that does not exceed 2 micrograms per square millimeter of balloon surface area. In a preferred embodiment, the first expandable device is a DEB loaded with a therapeutic agent at a concentration that does not exceed 1.5 micrograms per square millimeter of balloon surface area. In other embodiments, the first expandable device is a DEB loaded with a therapeutic agent at a concentration that does not exceed 0.75 micrograms per square millimeter of balloon surface area; and in yet other embodiments, the first expandable device is a DEB loaded with a therapeutic agent at a concentration that does not exceed 0.3 micrograms per square millimeter of balloon surface area. In embodiments relating to the treatment of peripheral arteries, the DEBs of the present invention, having as-manufactured dimensions of 7×40 mm or 6×40 mm, preferably comprise no more than 1,500 μg, 1,000 μg, 500 μg, or 300 μg of paclitaxel in total. This significant reduction in drug loading compared with conventional DEBs is due to the surprising observation that use of a second expandable device in conjunction with drug delivery from the first expandable devices of the present invention (e.g., DEBs) allow for long-term drug retention within the affected tissue that is significantly greater than retention following drug delivery from DEBs alone. While not wishing to be bound by theory, the inventors believe that the structure, configuration, chemical composition of material used in second expandable device and/or surface area of the second expandable device(s) of the present invention are responsible for this greatly enhanced drug retention. As demonstrated below, this surprising result occurs regardless of the order in which the first and second expandable devices of the present invention are deployed within the body.

Table II compares the tissue retention of paclitaxel in a sheep SFA model one hour (left) or seven days (right) after drug delivery under the following conditions: (I) one-minute delivery from a 7 mm×40 mm DEB loaded with 2638 micrograms of paclitaxel alone; (II) one-minute delivery from a 7 mm×40 mm DEB loaded with 2638 micrograms of paclitaxel, followed by implantation of a second expandable device of the present invention comprising 32 strands of 0.006 inch cross-sectional diameter; and (III) implantation of a second expandable device of the present invention comprising 32 strands of 0.006 inch cross-sectional diameter, followed by one-minute delivery from a 7 mm×40 mm DEB loaded with 2638 micrograms of paclitaxel.

TABLE II

| | Amount of | | Amount of | |
|---|---|---|---|---|
| | paclitaxel | Estimated % of | paclitaxel | Estimated % of |
| Delivery | in tissue | drug retained | in tissue | drug retained |
| Condition | after 1 hour | after 1 hour | after 7 days | after 7 days |
| I | 7.1 μg | 3% | <1 μg | 0.4% |
| II | 131 μg | 50% | 94 μg | 36% |
| III | 109 μg | 41% | 16 μg | 6% |

It should be noted that the estimated percentages of drug retained in the tissue are based upon an estimated drug transfer efficiency of 10% resulting from the approximately one-minute delivery of paclitaxel from the DEB (i.e., such that 264 micrograms of the paclitaxel loaded into the DEB is delivered into surrounding tissue during the approximately one-minute balloon expansion time). Given this surprising experimental data, the inventors have found it possible to significantly reduce drug loading rates in DEBs when used in conjunction with the second expandable devices of the present invention, as set forth herein, while still delivering effective drug doses that are retained in tissue for prolonged periods of time.

In certain embodiments, the strands used in the second expandable device of the invention comprise additives. In one example, such additives are neutralizing agents such as calcium salts (e.g., calcium carbonate or calcium phosphate), or other salts such as barium salts that increase the mechanical strength of the strands into which they are incorporated and further act to neutralize any acidic byproducts resulting from the degradation of the strand material(s). In another example, such additives are plasticizers such as polyethylene glycol (PEG) that dissolve from the strand(s) in vivo, thus increasing the flexibility of the strand(s) and the implant over time.

The implants of the present invention are preferably radiopaque such that they are visible using conventional fluoroscopic techniques. In one embodiment, radiopaque additives are included within the polymer material of one or more strands of the second expandable device 100. Examples of suitable radiopaque additives include particles comprising iodine, bromine, barium sulfate, and chelates of gadolinium or other paramagnetic metals such as iron, manganese, or tungsten. In another embodiment, the radiopaque groups, such as iodine, are introduced onto the polymer backbone. In yet another embodiment, one or more biostable or biodegradable radiopaque markers, preferably comprising platinum, iridium, tantalum and/or palladium are produced in the form of a tube, coil, sphere or disk, which is then slid over one or more strands of fiber to attach to the ends of device 100 or at other predetermined locations thereon. When the marker is in the form of a tube or coil, it has a preferable wall thickness of about 0.050 to 0.075 mm and a length of about 0.3 to 1.3 mm. The tube is formed by extrusion or other methods known in the art. The coil is formed by winding a wire around a mandrel of desired diameter and setting the coil with heat or other methods known in the art.

To facilitate delivery into a patient the second expandable device 100 is preferably loaded into a delivery catheter just prior to being implanted into a patient. Loading the device 100 in close temporal proximity to implantation avoids the possibility that the polymer of the device 100 will relax during shipping, storage, and the like within the delivery catheter and be unable to fully expand to a working configuration. As such, one aspect of the invention includes a method of delivering an implant of the invention that comprises the step of loading the implant into a delivery catheter within a short period of time, and preferably within one hour, before implantation into a body lumen. It should be noted, however, that it is not required that the implants of the present invention are loaded into delivery catheters just prior to being implanted. In fact, one advantage of the present invention is that it provides self-expanding implantable medical devices with preferred expansion characteristics and mechanical properties even after being loaded in a delivery catheter for prolonged periods.

While aspects of the invention have been described with reference to example embodiments thereof, it will be understood by those skilled in the art that various changes in form and details may be made therein without departing from the scope of the invention.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

All of the systems and/or methods disclosed and claimed herein can be made and executed without undue experimentation in light of the present disclosure. While the systems and methods of the present invention have been described in terms of preferred embodiments, it will be apparent to those of skill in the art that variations can be applied to the systems and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

Although the present disclosure has been described with reference to exemplary embodiments and implementations thereof, the disclosed systems and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope thereof.

We claim:

1. A method of treating a patient, comprising the steps of:
identifying a blood vessel with said patient for treatment, said blood vessel having an internal surface;
introducing a first expandable device into said blood vessel, said first expandable device comprising an outer surface;
contacting said internal surface of said blood vessel with at least a portion of said outer surface of said first expandable device at a target location of said blood vessel;
delivering a therapeutic agent to said internal surface of said blood vessel from said outer surface of said first expandable device;
withdrawing said first expandable device from said patient;
introducing a second expandable device into said blood vessel, said second expandable device comprising a tubular structure comprising a braided structure of at least one strand, said braided structure having an as-manufactured configuration comprising a surface area within a range of 100 to 500 square millimeters per 10 millimeters of tubular structure length;
expanding said second expandable device in said blood vessel;
contacting said internal surface of said blood vessel at said target location with a surface of said second expandable device; and
leaving said second expandable device implanted in said blood vessel.

2. The method of claim 1, wherein said outer surface of said first expandable device is at least partially coated with a coating that comprises said therapeutic agent.

3. The method of claim 2, wherein said first expandable device comprises a balloon having a surface area.

4. The method of claim 3, wherein said therapeutic agent is paclitaxel.

5. The method of claim 4, wherein an amount of paclitaxel is up to about 1.5 micrograms per square millimeter of said surface area of said balloon.

6. The method of claim 5, wherein a total amount of paclitaxel within said coating is less than 1,500 micrograms.

7. The method of claim 5, wherein a total amount of paclitaxel within said coating is less than 1,000 micrograms.

8. The method of claim 5, wherein a total amount of paclitaxel within said coating is less than 500 micrograms.

9. The method of claim 5, wherein a total amount of paclitaxel within said coating is less than 300 micrograms.

10. The method of claim 4, wherein the outer surface of said first expandable device is characterized by a surface area, and an amount of said paclitaxel within said coating does not exceed 1.5 micrograms per square millimeter of the surface area of said outer surface.

11. The method of claim 6, wherein said coating further comprises urea.

12. The method of claim 1, wherein said method further comprises the step of making an incision in said patient, and said first and second expandable devices are inserted through said incision.

13. The method of claim 1, wherein said therapeutic agent is selected from the group consisting of everolimus, sirolimus, zotarolimus, and biolimus.

14. The method of claim 1, wherein said second expandable device is self-expanding.

15. The method of claim 14, wherein said strand comprises a biodegradable material.

16. The method of claim 15, wherein said biodegradable material comprises poly(lactic acid co-glycolic acid).

17. The method of claim 16, wherein said strand is at least partially coated with a conformal coating.

18. The method of claim 17, wherein said conformal coating comprises poly(lactic acid-co-caprolactone).

19. The method of claim 1, wherein said step of introducing said expandable device into said blood vessel is carried out after said step of withdrawing said first expandable device from said patient.

20. The method of claim 1, wherein said step of introducing said first expandable device into said blood vessel is carried out after said step of contacting said internal surface of said blood vessel with said surface of said second expandable device.

21. The method of claim 1, wherein the surface area of said braided structure is within a range of 300-400 square millimeters per 10 millimeters of tubular structure length.

22. A kit, comprising:
a first expandable device having an outer surface, said outer surface characterized by a surface area;
a second expandable device comprising a tubular structure comprising a braided structure of at least one strand, said braided structure having an as-manufactured configuration comprising a surface area within a range of 100 to 500 square millimeters per 10 millimeters of tubular structure length;
wherein said outer surface of said first expandable device is at least partially coated with a coating that comprises paclitaxel, and an amount of said paclitaxel within said coating does not exceed 1.5 micrograms per square millimeter of the surface area of said outer surface.

23. The method of claim 22, wherein a total amount of paclitaxel within said coating is less than 1,500 micrograms.

24. The method of claim 22, wherein a total amount of paclitaxel within said coating is less than 1,000 micrograms.

25. The method of claim 22, wherein a total amount of paclitaxel within said coating is less than 500 micrograms.

26. The method of claim 22, wherein a total amount of paclitaxel within said coating is less than 300 micrograms.

27. The method of claim 22, wherein the surface area of said braided structure is within a range of 300-400 square millimeters per 10 millimeters of tubular structure length.
